(19) United States
(12) Patent Application Publication
(10) Pub. No.: US 2022/0152227 A1
(43) Pub. Date: May 19, 2022

(54) STICKY PARTICLES FOR EFFICIENT IMAGING

(71) Applicant: The Johns Hopkins University, Baltimore, MD (US)

(72) Inventors: Guanshu Liu, Nottingham, MD (US); Zheng Han, Greenbelt, MD (US)

(21) Appl. No.: 17/438,874

(22) PCT Filed: Mar. 13, 2020
(86) PCT No.: PCT/US2020/022832

§ 371 Date: Sep. 13, 2021

Related U.S. Application Data
(60) Provisional application No. 62/817,657, filed on Mar. 13, 2019.

(51) Int. Cl. A61K 49/18; A61K 49/08

(52) U.S. Cl. CPC A61K 49/1872; A61K 49/085; A61K 49/1812

(57) ABSTRACT

Described are compositions and methods of manufacturing extracellular vesicles comprising sticky imaging particles. In one aspect, methods include associating a sticky element to imaging particles forming sticky imaging particles. The sticky imaging particles are associated with extracellular vesicles (EV) to form a mixture comprising extracellular vesicles (EV) that comprise sticky imaging particles. The method also include separating the sticky imaging particles associated with EVs from sticky imaging particle that are not associated with EVs.

The present application claims the benefit under 35 U.S.C. 119 of U.S. provisional application No. 62/817,657, filed Mar. 13, 2019, which is incorporated herein by reference in its entirety.

### STATEMENT OF GOVERNMENTAL INTEREST

This invention was made with government support under grant nos. CA211087 and CA215860 awarded by the National Institutes of Health. The government has certain rights in the invention.

### BACKGROUND

Extracellular Vesicles (EV) are small membranous blebs or vesicles released from nearly all mammalian cell types and lower eukaryotes and prokaryotes, severing as important mediators of intercellular communication to regulate a diverse range of biological processes [1]. There are two types of EVs, exosomes (40-120 nm diameter), which derived from the endolysosomal pathway, and microvesicles (50-500 nm diameter), which are shed directly from the plasma membrane. By fusing with cells, exosomes can transfer a large body of receptors, proteins, genetic materials (including mRNA and microRNA) and lipids, shuttling information to target cells that are at immediate vicinity or at a distance [2]. Stem cell therapies, based on induced pluripotent stem cells (iPSCs) or mesenchymal stem cells (MSCs), have shown great promise in the treatment of multiple human diseases [3-6]. Exosomes secreted by stem cells have also garnered increasing attention, as exosomes are important components of the stem cell secretome [7]. The regenerative potential of stem cell-derived exosomes have been described in a number of diseases, such as cardiac diseases [8, 9] and kidney injury [10-13]. Therefore, exosomes are feasible cell-free substitutes of stem cell therapy.

In vivo tracking of exosomes through imaging is important for understanding the biodistribution and target cell interaction of administered exosomes. In clinics, verification of the localization of administered exosomes to diseased organ may be predictive of therapeutic responses. The pre-requisite of in vivo tracking is efficient labeling and purification of the exosomes with imageable probes. To date, very few studies have demonstrated the potential of optical imaging, single positron emission tomography (SPECT) and magnetic resonance imaging (MRI) of exosomes. For example, exosomes can be labeled with fluorescent dyes through electroporation and free dyes can be separated from exosomes due to their much smaller size. Fluorescence imaging has been used to assess the biodistribution of MSC-derived exosomes [14]. However, fluorescence imaging is less sensitive in detecting deep-tissue signals, therefore limiting its application for tracking accumulation of exosomes in deep organs. Luciferase-expressing exosomes can also be tracked in vivo through bioluminescence imaging [15], which, however, requires luciferin injection. Exosomes can also be labeled with radionuclide for radionuclide imaging [16]. However, the ionizing-radiation of radionuclide, along with their half-life restriction, also complex and increases the risk of the imaging procedure. MRI tracking of exosomes is a desirable modality due to the absence of ionizing radiation and its high soft-tissue resolution. However, owing to the inherently low sensitivity of MRI, to the best of our knowledge, there are only two studies showing the MRI tracking of superparamagnetic iron oxide particle (SPIO)-labeled EVs [17, 18]. In a report by Busato et al., SPIO particles were said to be introduced into the parent cells (adipose stem cells) and the EVs secreted from these cells were collected [18]. This approach does not require specific purification method to separate free SPIO from the produced EVs. However, the labeling efficiency is low. Hu et al. used electroporation to load EVs with SPIOs and subsequently treated them by ultracentrifugation at 100,000 g for 2 h [17]. The labeled EVs were said to be visualized in draining lymph nodes after being injected into the footpads of mice. However, ultracentrifugation may not be efficient to separate free SPIO and labeled EVs due to their similar size, which may result in low purification efficiency. Thus, efficient methods for labeling and purification of EVs with SPIO for in vivo tracking of these particles in MRI is still an unmet need.

### SUMMARY

We now provide methods for manufacturing extracellular vesicles comprising sticky imaging particles.

In one aspect, preferred methods include associating one or more sticky elements as specified herein to one or more imaging particles to form sticky imaging particles; and forming sticky imaging particles associated with extracellular vesicles (EV). In certain preferred embodiments, sticky imaging particles are incubated with extracellular vesicles (EV) to form sticky imaging particles associated with extracellular vesicles (EV). For instance, the associated stocky imaging particles may be incubated with extracellular vesicles (EV) to form stiocky imaging particles encapsulated in extracellular vesicles (EV).

In one aspect, the method includes attaching a sticky element that suitably binds to a chromatography material to imaging particles thereby forming sticky imaging particles. The sticky imaging particles are incubated with extracellular vesicles (EV) and a mixture is formed comprising sticky imaging particles associated with (e.g. encapsulated in) extracellular vesicles (EV) and sticky imaging particles not associated in such manner with extracellular vesicles (EV).

Sticky imaging particles encapsulated in EVs are suitably separated from the encapsulated sticky imaging particles. Imaging particles used in the present invention may comprise iron, gadolinium, manganese, copper, paramagnetic material, ferromagnetic material, diamagnetic material, or a combination thereof. An example of a preferred imaging particle is a supermagnetic iron oxide (SIPO) particle. Preferred imaging particles may have a size in the range of 2 to 10 nm, although particle sizes may suitably vary. In certain preferred aspects, the imaging particles of the present invention may be magnetic and/or nanoparticles. The sticky imaging particles associated with (e.g. encapsulated in) EVs are separated from sticky imaging particles that are not associated with EVs (e.g. unencapsulated sticky imaging particles) suitably by using a chromatography material. Examples of suitable chromatography materials used in the present invention include agarose, sepharose, or combinations thereof. In one aspect, the sticky element attached (e.g. bonded by covalent and/or non-covalent bonds) to the sticky imaging particles that are not associated with EVs adheres to the chromatography material while EVS that comprise sticky imaging particles are less adherent to the chromatography material to thereby provide separation of the materials. EV that comprise sticky imaging particles (e.g. the sticky imaging particles associated with (e.g. encapsulated in) EVs) can flow through a column of chromatography material more readily (faster rate) relative to sticky imaging particles that are not associated with EVs allowing the formation of a purified composition of sticky imaging particles that are not associated with EVs as well as purified composition of EVs that comprises sticky imaging particles (e.g. sticky imaging particles that are associated with EVs) as separated, purified compositions. Such separated or purified compositions then can be administered to patients as desired, or otherwise utilized as desired.

Suitable sticky elements of the present invention include peptide materials, for example a peptide having two or more amino acid residues and is some embodiments a peptide having a size in the range of 2 to 500 amino acids, or 2, 3, 4, 5, 6, 7, 8, 9, 10, 20 or 30 amino acids to 40, 50, 60, 70, 80, 90, 100, 120, 14, 160, 180, 200, 220, 240, 260, 280, 300, 320, 340, 360 380, 400, 420, 440, 46, 480 or 55 amino acids. In some embodiments, a sticky element include peptides that comprise, consist essentially of or consist of one or more histidine (HIS) amino acid residues. In other embodiments, a sticky element may include a peptide including 2 to 20 or more HIS amino acid residues.

In still other embodiments, the associating (includes e.g. attaching) step occurs by incubating the imaging particle in a fluid e.g. one or more solvents such as e.g. EDC, sulfo-NHS, PBS and then adding the sticky element.

In certain embodiments, in an incubating step, electroporation may be used to form the extracellular vesicles comprising the sticky imaging particles.

Examples of cells used in the present invention include for example induce pluripotent stem cells (iPSC), mesenchymal stem cell (MSCs), neural stem cell (NSC), hematopoietic stem cell (HSC), or a combination thereof.

Another embodiment of the present invention are sticky imaging particles comprising an imaging particle comprising a sticky element that binds to a chromatography material. In one embodiment, a sticky imaging particle may comprise a sticky element that is a peptide comprising two or more amino acid residues. Examples of peptides used as sticky elements may comprise, consist essentially of or consist of histidine (HIS) amino acid residues and/or have a size in the range of 2 to 20 HIS amino acid residues.

Another embodiment of the present invention is an imaging composition comprising EV encapsulated sticky imaging particles. Preferred imaging compositions of the present invention suitably have greater than 40 weight %, 50 weight %, 60 weight %, 70 weight %, 80 weight %, 85 weight %, 90 weight %, 95 weight %, or 97 weight % or more of sticky imaging particles associated (e.g. encapsulated) with extracellular vesicles (EV), where such weight % are based on total weight of the imaging composition.

In certain aspects, preferred are composition of sticky imaging particles associated with EVs and having less than 3%, 5%, 10% or 15% sticky imaging particles that are not associated or encapsulated with sticky imaging. In some embodiments, compositions of the present invention are pharmaceutical compositions.

In other aspects, methods of imaging are provided. In preferred aspects, such methods may comprise the step of administering an imaging composition as disclosed herein to a subject. Following administration, preferably the imaging composition is allowed to reach its target site such as a particular organ, cell or tissue. Energy is suitably applied to the target site and the imaging composition is identified at the target site. A target site maybe a cell, tissue, or organ of a subject. An example of energy applied to a target includes radio frequency pulse, ionizing radiation, exciting photon, or combination thereof. The imaging composition, including the EV encapsulated sticky imaging particles, is identified by viewing a MRI, X-ray/CT, nuclear imaging, or by optical imaging, as examples.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “chromatography material” is meant a material for the separation of different components of a mixture by the different travel speeds of the components through the material.

By “disease” is meant any condition, disorder that damage, or interfere with the normal function of a cell, tissue, or organ. Examples of diseases include cancer or organ damage.

By “EDC” is meant 5-Ethynyl-2′-deoxycytidine.

By “effective amount” without further limitation can mean herein the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “imaging-effective amount” is meant an amount (such as an amount of an sticky element imaging composition) that can enable desired visualization of an administered composition.

By “EV” is meant extracellular vesicles.

By “express” is meant the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “imaging particle” is meant a particle, typically a nanoparticle, comprising iron, gadolinium, manganese, copper, paramagnetic material, ferromagnetic material, diamagnetic material, iodinated polymer, gold particle, fluorescent particle, radioactive particle, or a combination thereof. The imaging particle can generate contrast in imaging modalities, including magnetic resonance imaging (MRI), computed tomography (CT), positron emission tomography (PET), single photon emission tomography (SPECT), optical imaging, etc.

By “LPS” is meant lipopolysaccharide, an endotoxin derived from the outer membrane of gram-negative bacteria.

By “nanoparticle” is meant a particle having a size in the range of 1 nm to 100 nm.

By “obtaining” as in “obtaining an agent” is meant synthesizing, purchasing, or otherwise acquiring the agent.

By “polyhistidine-tag” or “HIS tag” is meant an amino acid motif in proteins that consists of at least six histidine residues (typically between one to six histidine residues), often at the N- or C-terminus of the protein. It is also known as hexa histidine-tag, 6× His-tag, His6 tag and by the trademarked name His-tag.

By “polypeptide,” “peptide” and “protein” terms used interchangeably is meant a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “purity” is meant the ratio of imaging particles that are associated with (e.g. encapsulated in) EVs with respect to the total imaging particles in the solution. For example, the imaging compositions of the present invention have greater than 85%, 90%, 95%, or 97% of sticky imaging particles. In certain aspects, purity also can be is defined as a composition of encapsulated sticky imaging particles having less than 3%, 5%, 10% or 15% unencapsulated sticky imaging particles.

By “reduces” is meant a negative alteration of at least 10%, 25%, 50%, 75%, or 100%.

By “reference” is meant a standard or control conditions such as a sample (human cells) or a subject that is a free, or substantially free, of an agent such as one or more EVs encapsulating a sticky magnetic particle of the present invention.

By “reference sequence” is meant a defined sequence used as a basis for sequence comparison. A reference sequence may be a subset of or the entirety of a specified protein or nucleic acid sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “SPIO” is meant a superparamagnetic iron oxide particle, typically a nanoparticle.

By “sticky element” is meant a composition or material that 1) can bind to a chromatography material such as a binding resin differentially such as to permit separation as disclosed herein and 2) is capable of being associated with (e.g. attached e.g. by covalent and/or non-covalent bond) to an imaging particle as disclosed herein. Examples of sticky elements include for instance a polyhistidine tag, biotin/streptavidin, antibody/antigens, or click chemistry conjugating motifs, glutathione S-transferase (GST), as well as other tags for chromatography purification.

By “sticky imageable particle” is meant a composition of the present invention including an imaging particle comprising (or attached to e.g. by covalent and/or non-covalent bond) a sticky element.

By “subject” or “patient” is meant any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith or imaging of an organ or tissue. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

### BRIEF DESCRIPTION OF THE DRAWINGS

FIG. 1A-1G. Preparation magneto-EV and characterization of purified magneto-EVs. A. Schematic illustration of the preparation of SPIO his-tag (SPIO-His), by conjugating hexahistidine (6× His-tag) polypeptide to the carboxyl groups of SPIO particles using EDC (1-Ethyl-3-(3-dimethylaminopropyl)-carbodiimide), and NHS (sulfo-N-hydroxysuccinimide) chemistry. B. As a result from the high affinity between the His-peptide and nickle ion, the SPIO-His particles bind to Ni²⁺ immobilized on beads (e.g., Ni-NTA resins) for further purification. C. Schematic illustration of the encapsulation of SPIO-His into EVs by electroporation and subsequent purification by removing unencapsulated SPIO-His from the elute using Ni-NTA affinity chromatography. D. Size distribution as measured by dynamic light scattering (DLS) for SPIO-His, EVs, SPIO-His/magneto-EV/EV mixtures after electroporation, and the final purified elute, respectively. E. TEM images of EV, SPIO-His and EV-SPIO, respectively. F. Concentration of magneto-EVs using a magnet. Eluted magneto-EV solution was placed on a magnet overnight to pellet magneto-EVs. The photograph shows the pelleted magneto-EV at the bottom of a microcentrifuge tube. G. T₂-weighted (T₂w) images of magneto-EVs at different concentrations, unlabeled EVs, and PBS. Mean R₂ values of magneto-EVs are plotted with respect to their concentration, from which the r₂ (relaxivity) was estimated.

FIG. 2A-2J. MRI detection of the uptake of magneto-EV in control and injured kidneys in the LPS-AKI model. A. Timeline of preparation of animal model and injection of EVs. B. T₂*-weighted (T₂*w) images in a representative normal control mouse before and at 5 and 30 min after i.v. injection of magneto-EVs and SPIO-His, respectively. C. Corresponding contrast enhancement maps, defined as ΔT₂*w=T₂*w (post)−T₂*w (pre). D. Mean dynamic signal change in the control kidneys (n=6). E. T₂*-weighted (T₂*w) images in a representative LPS-AKI mouse before and at 5 and 30 min after the i.v. injection of magneto-EVs and SPIO-His, respectively. F. Corresponding contrast enhancement maps. G. Mean dynamic signal change in the injured kidneys (n=6). H. ΔR₂*(1/T_{E}×ln(SI^{post}/SI^{pre})) maps at 30 min after injection of magneto-EVs. I. Quantitative comparison of mean kidney ΔR₂* values at 30 min between different groups. *, P<0.05, ns: not significant, n=6, unpaired two-tailed Student's t-test. J. Quantitative comparison of mean liver ΔR₂* values at 30 min between different groups. *, P<0.05, ns: not significant, n=6, unpaired two-tailed Student's t-test.

FIG. 3A-3C. Dynamics of ΔR₂* contrast change in kidney and liver generated by magneto-EVs. A. Coronal T₂*w image of a representative mouse showing the ROI selection for kidneys and liver. Dynamic changes of ΔR₂*, calculated by 1/T_{E}*×ln(Sₚₒₛₜ/Sₚᵣₑ), of kidney (B) and liver (C) of normal and LPS-AKI mice after magneto-EV or SPIO-His injection.

FIG. 4A-4K. Biodistribution of magneto-EVs in the injured kidney and their therapeutic effect in the LPS-AKI model. A. Different biodistribution patterns of magneto-EVs (left) and SPIO-His (middle) in representative LPS-AKI kidneys, as revealed by ex vivo high-resolution MRI. Image of a representative non-injected kidney on the right is shown as a control reference. B. 3D reconstruction of a representative kidney showing the biodistribution of magneto-EVs (gold-colored dots). Blood vessels are shown in purple color (see also Supplementary Video 3). C. Quantitative comparison of the relative hypointense areas (%) in the cortex of AKI mice injected with magneto-EVs, SPIO-His, or without injection (****: P<0.0001, unpaired Student's t-test, n=15). D. Prussian blue stains showing the distribution of magneto-EVs and SPIO in the injured kidney (Left: whole kidney; Right: zoom-in; Blue=SPIO; Red=nucleus). E. VCAM-1 staining of a representative kidney showing extensive inflammation (green) occurring in the cortex. Tissue was counterstained with DAPI (blue). F. Periodic acid-Schiff (PAS) staining of the section corresponding to the Prussian blue stain on the left. G. Biodistribution of magneto-EVs (left) and SPIO-His (right) in a representative normal control kidney. H. Prussian blue stains showing the distribution of magneto-EVs and SPIO-His in uninjured kidney (Left: whole kidney; Right: zoom-in; Blue=SPIO; Red=nucleus). I. Survival curves of AKI mice treated with 2×10⁹ iPSC-EVs at different time points (0, 3, and 24 hours) and vehicle control (PBS). A significant treatment response can be observed at 0 hours, i.e. co-injection of LPS and iPSC-EV (**: P=0.0023 vs. PBS, log-rank test). J. Ex vivo MR images of a representative LPS-AKI kidney 30 min after injection 2×10⁹ FBS-derived magneto-EVs. K. Survival curves of LPS-AKI mice co-injected with iPSC-EV, FBS-EV, or PBS, respectively. **: P=0.0023, iPSC-EV vs. PBS; P=0.0025, iPSC-EV vs. FBS-EV; log-rank test.

FIG. 5. Hematoxylin-eosin (HE) stain of a representative section of LPS-AKI kidney, in which hemorrhages in the cortex of the kidney are indicated by black arrows.

FIG. 6A-6F. MRI tracking of i.v. administered magneto-EVs in the IRI-AKI model. A. Schematic illustration of the experimental IRI-AKI model and MRI acquisition. B. Representative in vivo T₂* image and ΔR₂* map at 30 min after EV injection. C. Dynamic ΔR₂* MRI signal changes in IRI and non-operated control kidneys (n=3 in each group). D. Comparison of ΔR₂* (30 min) values in IRI and control kidney for each mouse (*: P=0.04, two-tailed paired Student's t-test, n=3). E. Ex vivo high-resolution T₂*w MR image of a representative IRI kidney and unoperated kidney. F. Corresponding Prussian blue staining (Left: whole kidney; Right: zoom-in; Blue=SPIO; Red=nucleus.

FIG. 7A-7G. MRI tracking of magneto-EV accumulation in the IR heart. A. Schematic illustration of the experimental IR heart model and MRI acquisition. B. Macrophotograph of the heart with the IR region (arrow). C. Sagittal in vivo MR images of the heart. Yellow box indicates the slice position of the short-axis view. Short-axis pre- and post-injection in vivo T₂*w images (D) and enhancement maps, defined as ΔT₂*w=T₂* (post)−T₂*w (pre) (E) showing hypointense areas in the injured region around the apex of the heart. F. Ex vivo heart MR image showing accumulation of magneto-EVs (red arrow). G. Prussian blue staining of the injured heart (Left: whole heart; Right: zoom-in of sections 1-3).

### DETAILED DESCRIPTION

In preferred aspects, we have now an efficient labeling and purification method for preparing sticky magnetic particles for imaging including sticky SPIO particles comprising a sticky element such as a HIS tag. In one preferred system, sticky magnetic particle-loaded iPSC-derived exosomes were formed, which allows the non-invasive tracking of therapeutic exosomes in the treatment of organ disease such as acute kidney injury, as an example. After being encapsulated in EVs, sticky magnetic particles of the present invention can be considered in one aspects as “protected” inside the vesicles and lose contact with a chromatography material such as a binding resin located on the exterior surface of the EVs. In one preferred system, EVs comprising the sticky magnetic particles were successfully used to reveal the targeting properties of iPSC-derived EVs in injured mouse kidneys.

Embodiments of the disclosure concern methods and/or compositions for imaging, including imaging of organ damage directly or indirectly. In certain embodiments, individuals with organ damage such as injured kidneys are administered with EVs that encapsulate sticky magnetic particles of the present invention, and in specific embodiments an individual with organ damage is provided a sticky SPIOs encapsulated in EVs that have been purified by the methods of the present invention.

In particular embodiments of the disclosure, an individual is given EVs associated with (e.g. encapsulating) a sticky magnetic particle of the present invention in addition to the one or more pharmaceutical agents that may also be associated with (e.g. encapsulated in) the EV comprising the sticky magnetic particle. When combination therapy is employed with EVs associated with a sticky magnetic particle the additional therapy may be given prior to, at the same time as, and/or subsequent to the EVs associated with a sticky magnetic particle.

### Pharmaceutical Preparations

Pharmaceutical compositions of the present invention comprise an effective amount of one or more EVs associated with (e.g. encapsulating) a sticky magnetic particle such as SPIO comprising a sticky element such as a HIS tag, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one EV associated with (e.g. encapsulating) a sticky magnetic particle or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The EVs associated with (e.g. encapsulating) a sticky magnetic particle may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The EVs associated with (e.g. encapsulating) a sticky magnetic particle may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, compositions of the present invention suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include a sticky magnetic particle, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, sticky magnetic particles of the present invention may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, the EVs associated with a sticky magnetic particle are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet. In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, EVs associated with (e.g. encapsulating) a sticky magnetic particle may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compound EVs associated with a sticky magnetic particle may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, EVs associated with a sticky magnetic particle (for example, SPIO including a HIS tag) may be comprised in a kit.

The kits may comprise a suitably aliquoted EVs associated with a sticky magnetic particle and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the EVs encapsulating a sticky magnetic particle and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The EVs associated with (e.g. encapsulating) a sticky magnetic particle composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

### REFERENCES CITED ABOVE

1. Karpman, D., A. L. Stahl, and I. Arvidsson, Extracellular vesicles in renal disease. Nat Rev Nephrol, 2017. 13(9): p. 545-562.
2. Colombo, M., G. Raposo, and C. Thery, Biogenesis, Secretion, and Intercellular Interactions of Exosomes and Other Extracellular Vesicles. Annual Review of Cell and Developmental Biology, Vol 30, 2014. 30: p. 255-289.
3. Nori, S., et al., Long-term safety issues of iPSC-based cell therapy in a spinal cord injury model: oncogenic transformation with epithelial-mesenchymal transition. Stem Cell Reports, 2015. 4(3): p. 360-73.
4. Ye, L., et al., Induced pluripotent stem cells offer new approach to therapy in thalassemia and sickle cell anemia and option in prenatal diagnosis in genetic diseases. Proceedings of the National Academy of Sciences of the United States of America, 2009. 106(24): p. 9826-9830.
5. Robinton, D. A. and G. Q. Daley, The promise of induced pluripotent stem cells in research and therapy. Nature, 2012. 481(7381): p. 295-305.
6. Xu, D., et al., Phenotypic correction of murine hemophilia A using an iPS cell-based therapy. Proceedings of the National Academy of Sciences of the United States of America, 2009. 106(3): p. 808-813.
7. Santoso, M. R. and P. C. Yang, Molecular Imaging of Stem Cells and Exosomes for Myocardial Regeneration. Current Cardiovascular Imaging Reports, 2017. 10(11).
8. Adamiak, M., et al., Induced Pluripotent Stem Cell (iPSC)-Derived Extracellular Vesicles Are Safer and More Effective for Cardiac Repair Than iPSCs. Circulation Research, 2018. 122(2): p. 296-309.
9. Liu, B., et al., Cardiac recovery via extended cell-free delivery of extracellular vesicles secreted by cardiomyocytes derived from induced pluripotent stem cells. Nature Biomedical Engineering, 2018: p. 1.
10. Collino, F., et al., AKI Recovery Induced by Mesenchymal Stromal Cell-Derived Extracellular Vesicles Carrying MicroRNAs. J Am Soc Nephrol, 2015. 26(10): p. 2349-60.
11. Reis, L. A., et al., Bone marrow-derived mesenchymal stem cells repaired but did not prevent gentamicin-induced acute kidney injury through paracrine effects in rats. PLoS One, 2012. 7(9): p. e44092.
12. Bruno, S., et al., Mesenchymal stem cell-derived microvesicles protect against acute tubular injury. J Am Soc Nephrol, 2009. 20(5): p. 1053-67.
13. Bruno, S., et al., Microvesicles Derived from Mesenchymal Stem Cells Enhance Survival in a Lethal Model of Acute Kidney Injury. Plos One, 2012. 7(3).
14. Grange, C., et al., Biodistribution of mesenchymal stem cell-derived extracellular vesicles in a model of acute kidney injury monitored by optical imaging. Int J Mol Med, 2014. 33(5): p. 1055-63.
15. Imai, T., et al., Macrophage-dependent clearance of systemically administered B16BL6-derived exosomes from the blood circulation in mice. J Extracell Vesicles, 2015. 4: p. 26238.
16. Hwang, D. W., et al., Noninvasive imaging of radiolabeled exosome-mimetic nanovesicle using Tc-99m-HMPAO. Scientific Reports, 2015. 5.
17. Hu, L., S. A. Wickline, and J. L. Hood, Magnetic resonance imaging of melanoma exosomes in lymph nodes. Magn Reson Med, 2014.
18. Busato, A., et al., Magnetic resonance imaging of ultrasmall superparamagnetic iron oxide-labeled exosomes from stem cells: a new method to obtain labeled exosomes. Int J Nanomedicine, 2016. 11: p. 2481-90.
19. Zhang, J., et al., Triazoles as T2-exchange MRI contrast agents for the detection of nitrilase activity. Chemistry, 2018.

The following non-limiting examples are illustrative of the invention.

### EXAMPLE

### Example 1

### Magnetic Labeling of EVs and Purification

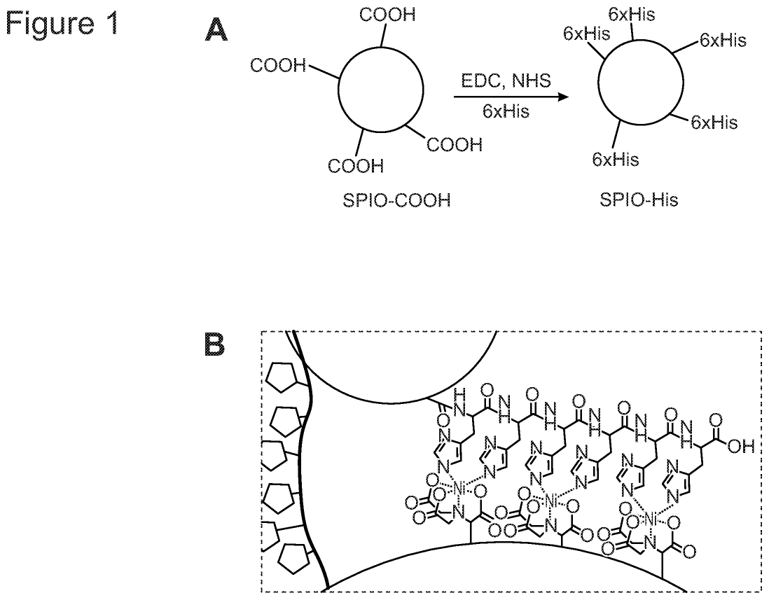

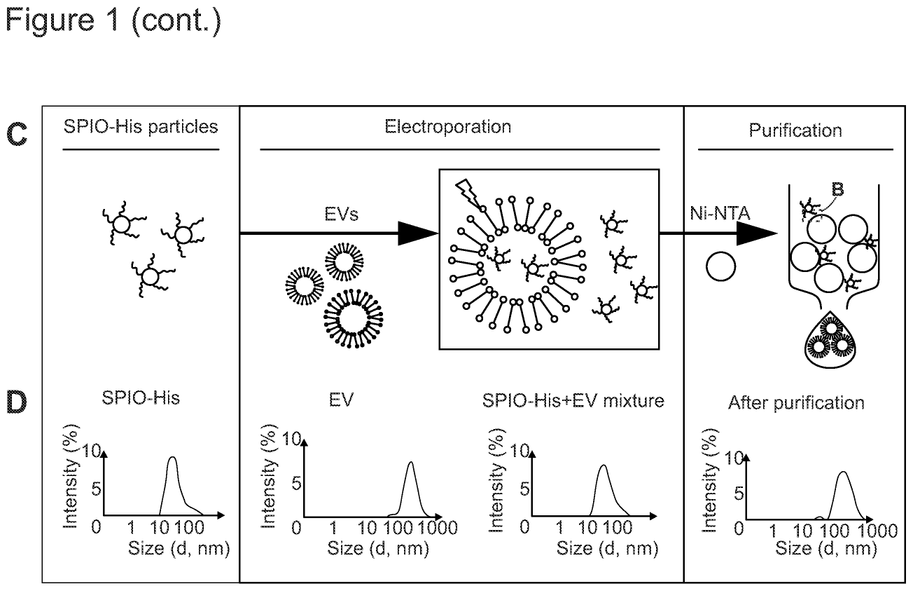

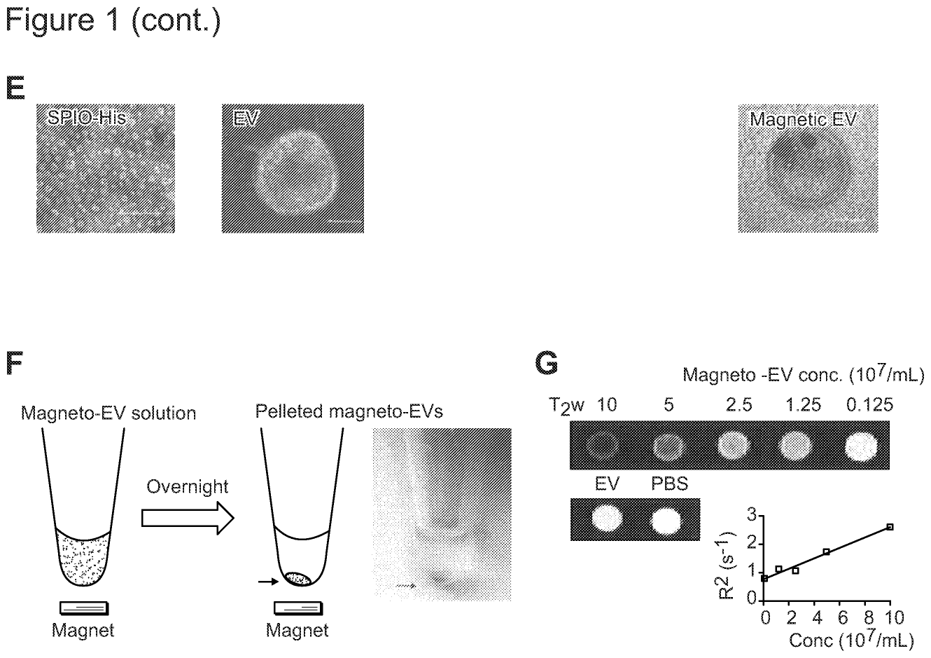

To prepare “sticky” magnetic nanoparticles, we first synthesized surface-modified SPIO nanoparticles by conjugating them with hexa-histidine peptides (6× His) using the synthetic route shown in FIG. 1A. With His-tags on the surface, which was confirmed by Fourier-transform infrared spectroscopy (Fourier-transform infrared spectrum of SPIO-His compared to that of SPIO—COOH, which shows the characteristic peak of SPIO-His near 1680 cm⁻¹, confirming the formation of amide bonds between carboxyl group and the amine group of histidine peptide), SPIO nanoparticles can selectively bind to Ni²⁺ immobilized nitrilotriacetic acid (NTA) agarose resins (Ni-NTA) (FIG. 1B). This complex has a distinct color, where the Ni-NTA column eluted with SPIO—COOH (no His tag) showed no color change whereas the one eluted with SPIO-His changed from light blue to brown.

The labeling and purification procedures of magneto-EVs are illustrated in FIG. 1C. In brief, the synthesized SPIO-His nanoparticles were first loaded into purified EVs by electroporation as described previously(22). The resulting solution containing a mixture of free SPIO-His and magneto-EVs was purified using a Ni-NTA column. Quantitative analysis of iron content in the solution pre- and post-elution revealed that the Ni-NTA column was able to remove 97.4% of unincorporated SPIO-His with minimal loss of EVs (˜5.4% as measured by nanoparticle tracking analysis). The average size was measured using dynamic light scattering (DLS) to be 43.9±16.5 nm, 248.2±107.2 nm, 45.6±15.9 nm, and 292.8±113.0 nm, for SPIO-His, EVs, unpurified magneto-EVs, and purified magneto-EVs, respectively (FIG. 1D). The size distribution of magneto-EVs closely resembled that of unlabeled EVs, indicating the efficient removal of unencapsulated SPIO-His particles. Labeling and purification were verified by transmission electron microscopy (TEM) (FIG. 1E), which showed no unencapsulated SPIO-His particles in the purified solution. TEM demonstrated that many EVs contained multiple SPIO-His particles. Moreover, the incorporation of magnetic particles allowed enrichment of magneto-EVs using a magnetic force (FIG. 1F).

In vitro MRI confirmed the hypointense contrast of magneto-EVs (FIG. 1G). At 9.4 T and 37° C., the R₂ enhancement was determined to be 1.83 s⁻¹ per 10⁸/mL EVs, which is equivalent to an r₂ relaxivity of 1.1×10¹⁰ s⁻¹mM⁻¹ per EV (10⁸/mL EVs=16.7 pM) or 659 s⁻¹mM⁻¹ Fe (10⁸/mL EVs contain 155 ng/mL Fe as measured by ICP-OES). From this, we estimated the in vivo detection limit to be approximately 8.76×10⁷ EVs/mL EVs kidney tissue (having an inherent R₂ of 32.03 s⁻¹ at 3T(24)), assuming a 5% MRI signal change.

In Vivo MRI of iPSC-EV Utake in an Injured Kidney Model

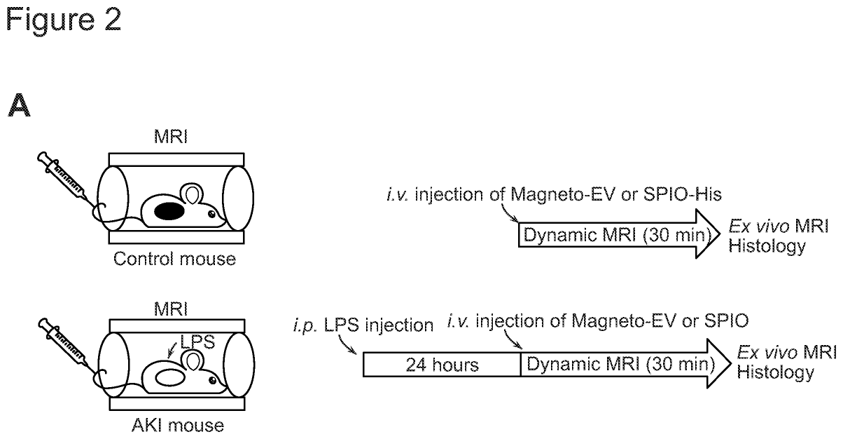

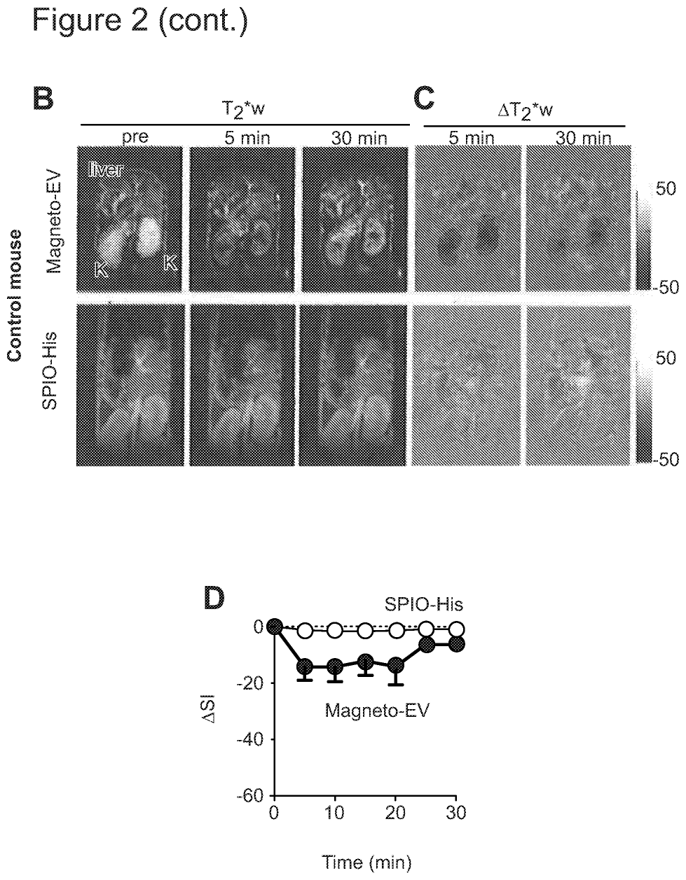

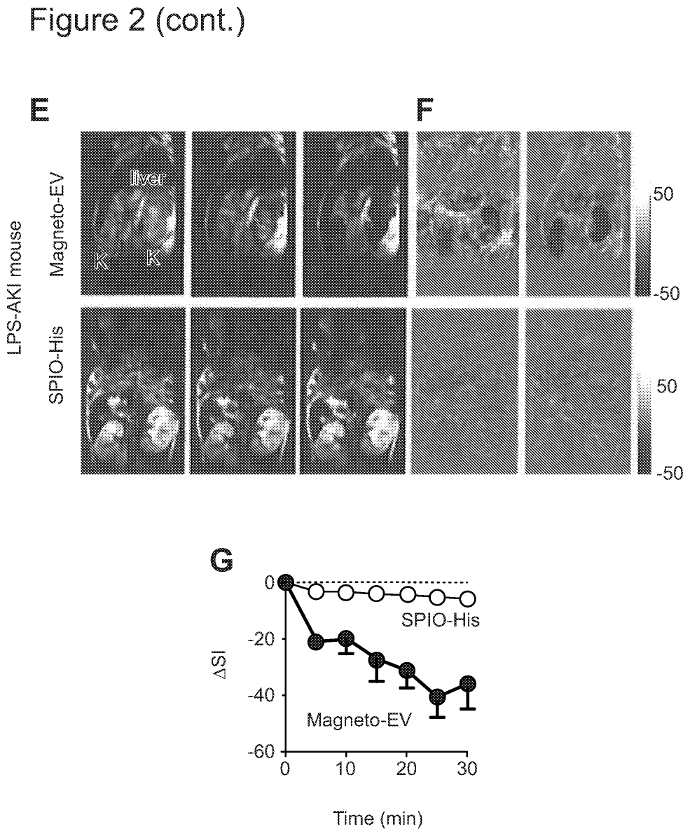

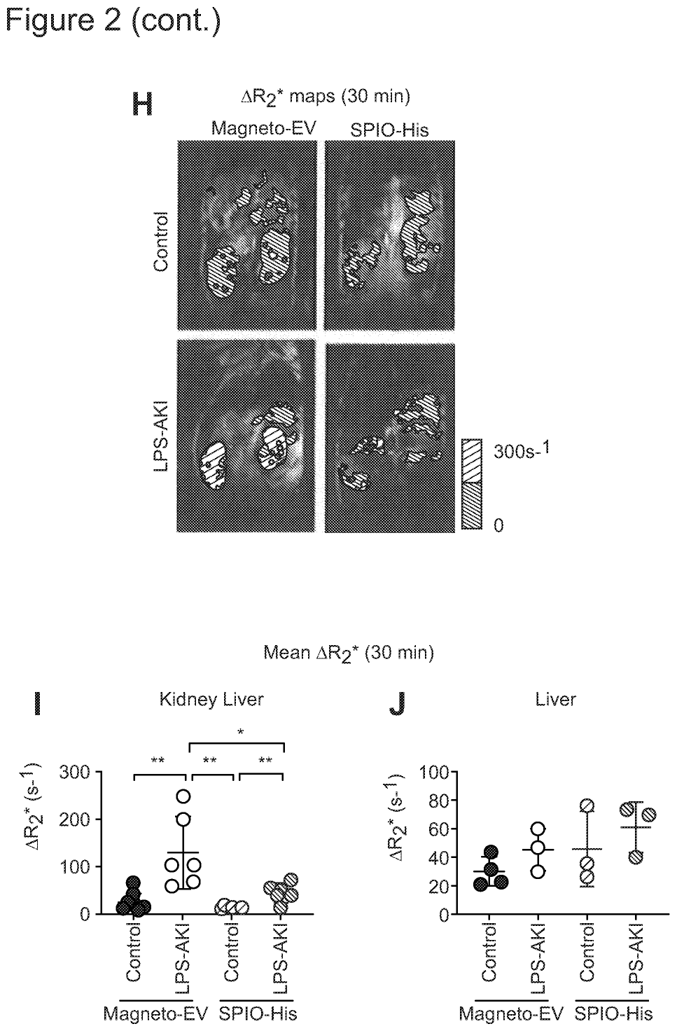

We first assessed the distribution of magneto-EVs in an lipopolysaccharides (LPS)-induced acute kidney injury (AKI) model, a well-established rodent model associated with severe systemic inflammation and irreversible kidney damage within 24-48 hours(25, 26). As SPIO nanoparticles strongly distort the local magnetic field and result in a much quicker T₂* decay of protons in nearby water molecules, the dynamic uptake of SPIO-containing magneto-EVs could be readily detected using T₂*-weighted (T₂*w) MRI in which magneto-EVs appear as hypointense spots. As shown in FIG. 2A, either SPIO-His particles or magneto-EVs were i.v. injected into uninjured control or LPS-AKI mice and the MRI signal was monitored dynamically for 30 minutes after injection. The 30-minute window was chosen because the blood half-life of EVs has been reported to be 2-4 minutes only(27).

For uninjured control mice, the MRI signal intensity in the kidney exhibited a rapid decrease immediately after i.v. injection of magneto-EVs and then remained stable between the 5 to 20 min, finally recovered to baseline at 20-30 min (FIGS. 2B-D). In contrast, when SPIO-His nanoparticles were injected at the same iron dose, no detectable signal change in the kidney could be observed. In the LPS-AKI mice (FIGS. 2E-G), a substantially different uptake pattern was observed in animals injected with magneto-EVs, with the MRI signal continuing to decrease for ˜25 min when it reached a plateau, indicating a continuous uptake in the injured kidneys. At 30 min after magneto-EV injection, the injured kidneys nearly lost all their signal, indicating a high uptake of magneto-EVs. LPS-AKI mice injected with SPIO-His nanoparticles alone showed only negligible MRI signal changes in the kidney, similar to the mice in the uninjured control group injected with SPIO-His particles.

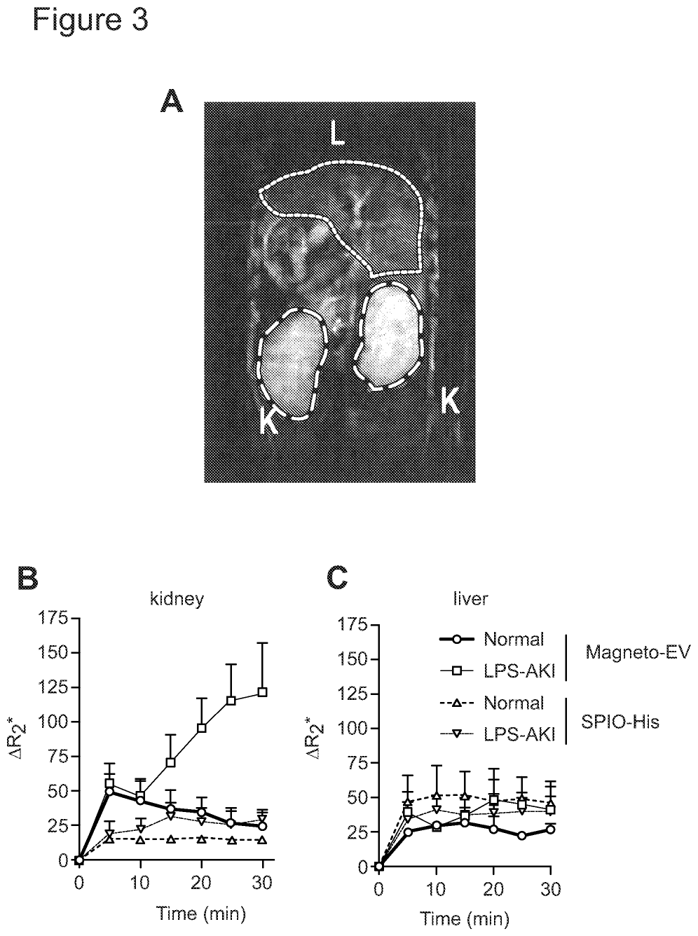

The amount of magneto-EVs or SPIO-His particles accumulated in the kidney was then quantitatively estimated using the changes of R₂* signal in the kidney, defined as ΔR₂*=1/T₂*(post)−1/T₂*(pre)=1/T_{E}×ln(SI^{post}/SI^{pre}), a commonly used metric in SPIO-enhanced MRI(28). Snapshot ΔR₂* contrast enhancement maps and changes at 30 minutes after magneto-EV injection are shown in FIG. 2H. A higher amount of magneto-EVs accumulated in injured vs. uninjured control kidneys. Weaker ΔR₂* contrast enhancement was seen in both groups of kidneys injected with SPIO-His nanoparticles. A quantitative comparison of kidney ROI values revealed a significantly higher ΔR₂* for the LPS-AKI group compared to uninjured controls (130.1 vs 24.69 s⁻¹, P=0.0024) (FIG. 2I). There was also a significant difference between the LPS-AKI mice injected with magneto-EVs vs. SPIO-His particles (130.1 vs. 46.0 s⁻¹, P=0.0254). The ΔR₂* in the LPS-AKI group after SPIO-His injection was also higher than that in the normal control group (46.0 vs, 14.3 s⁻¹, P=0.0035). In addition, we calculated the dynamic ΔR₂* values in the kidney for each group (FIG. 3B), confirming the differential kidney uptake dynamics for LPS-AKI mice injected with magneto-EVs versus all other groups.

To determine whether magneto-EVs can be taken up by the liver non-specifically, as is the case for most i.v.-injected SPIO nanoparticles, we further analyzed liver contrast-enhancement in both LPS-AKI and uninjured control mice (FIG. 3A). As shown in FIG. 1J, none of the groups showed significantly different ΔR₂* values at 30 min after injection of magneto-EVs or SPIO-His particles. Similar dynamic liver uptake patterns were observed among these groups (FIG. 3C), suggesting that there is negligible non-specific liver uptake of iPSC-EVs.

High-Resolution Ex Vivo MRI of iPSC-EV Uptake in an Injured Kidney Model

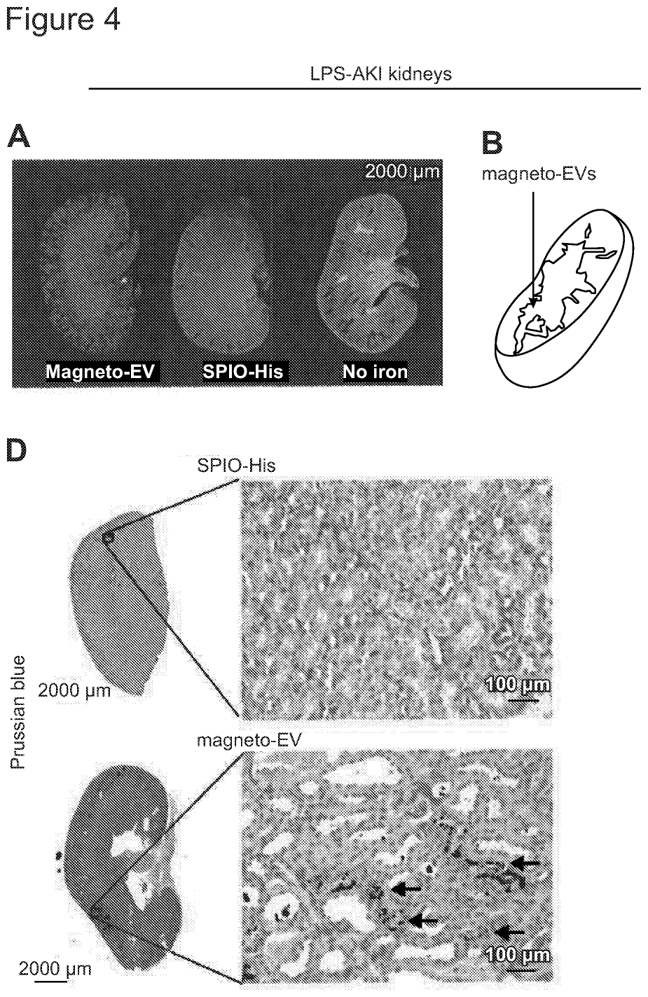

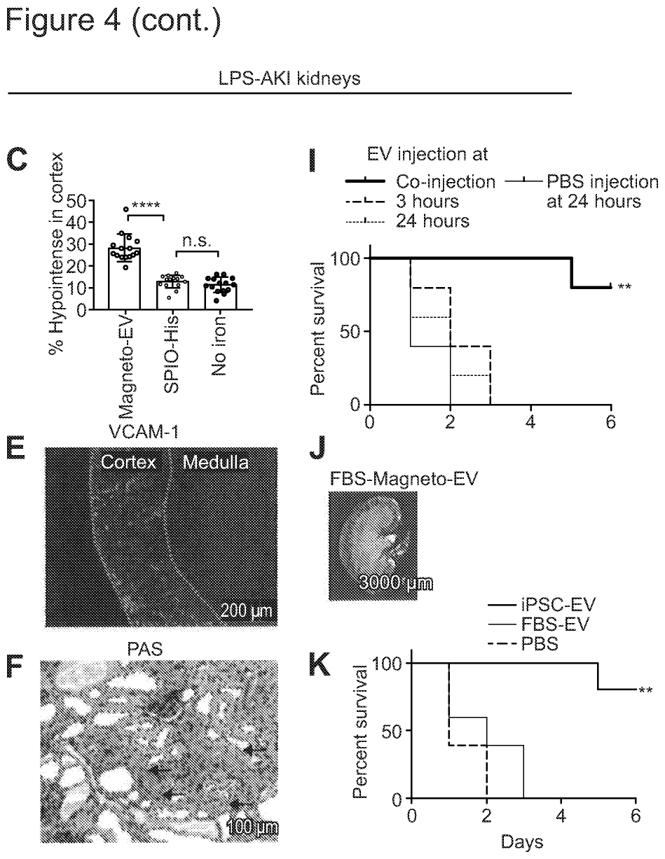

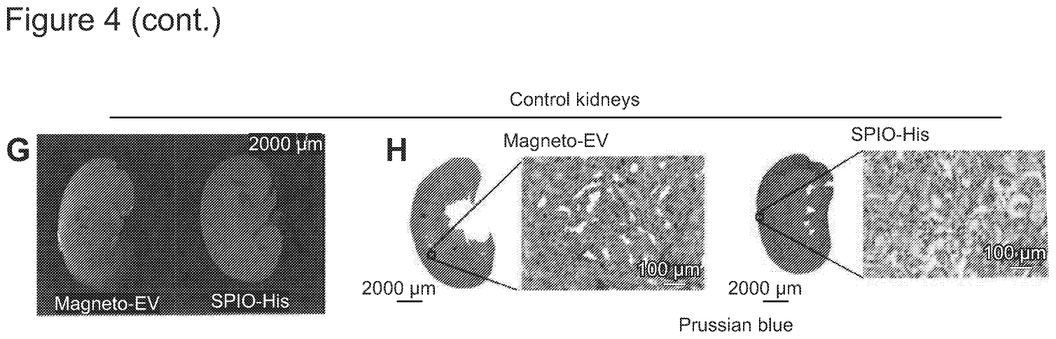

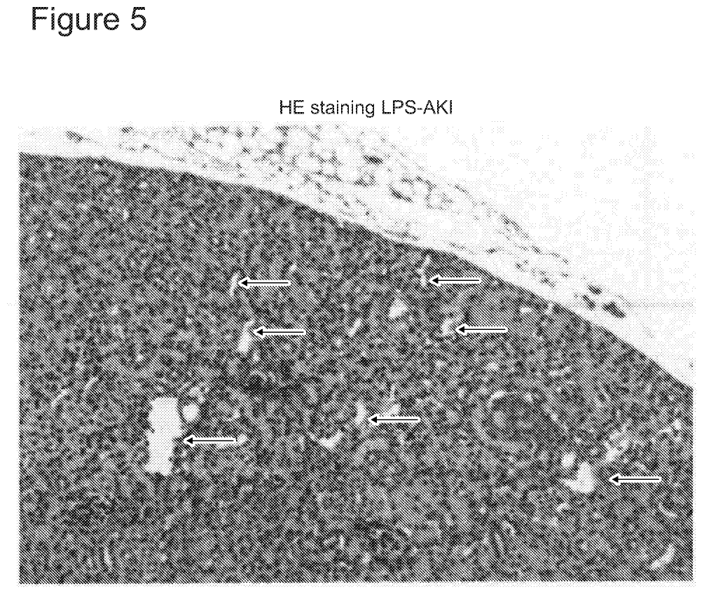

To study the biodistribution of magneto-EVs and naked SPIO-His particles in kidneys in more anatomic detail, we performed high resolution three-dimensional (3D) ex vivo MRI on fixed kidney samples that were excised at 30 min after magneto-EV or SPIO-His injection. FIG. 4A shows representative kidney T₂*w images of LPS-AKI mice injected with magneto-EVs, SPIO-His particles, and saline, respectively. Only the kidneys from mice receiving magneto-EVs demonstrated a high number of hypointense spots and streaks dispersed throughout the renal cortex (FIG. 4A, left). In contrast, kidneys of mice injected with SPIO-His particles showed far fewer black spots (FIG. 4A, middle), similar to the kidney without SPIO-His injection (FIG. 4A, right). The presence of black hypointensities in the last two groups may be due to the LPS-induced hemorrhage (H&E stain, FIG. 5). 3D reconstruction of hypointense voxels revealed that magneto-EVs distributed throughout the whole kidney, with preferential accumulation in the cortex (FIG. 4B). Quantitative analysis showed that approximately 28.1% of the cortex of AKI mice injected with magneto-EVs contained hypointensities (FIG. 4C), significantly higher than those injected with SPIO-His particles alone (12.5%, P<0.0001) or saline (11%, P<0.0001). Accumulation of magneto-EVs in the cortex was confirmed by Prussian blue staining for iron (FIG. 4D), where the tissue distribution of magneto-EVs showed a good agreement with that seen on ex vivo MRI. Furthermore, immunostaining for VCAM-1, a vascular inflammation marker, showed that LPS-AKI kidneys exhibited higher VCAM-1 expression in the cortex compared to the medulla (FIG. 4E). Prussian blue-positive iron also co-localized with dilated proximal tubules as shown on Periodic acid-Schiff (PAS) staining (FIG. 4F), with the tubular damage being a hallmark of the LPS-AKI model(29). In contrast, kidneys from control mice injected with either magneto-EVs or SPIO-His particles exhibited fewer hypointense areas, and those hypointense areas on T₂*w images (FIG. 4G) correlated well with the negative Prussian blue staining (FIG. 4H).

The accumulation of iPSC-derived EVs in the injury sites resulted in an observable therapeutic effect. A single dose of 2×10⁹ EVs was injected at 0 (LPS co-injection), 3, or 24 h after LPS injection (FIG. 4I). Compared to vehicle control (PBS), only the magneto-EV/LPS co-injection resulted in a statistically significant improvement of survival rate (n=5, P=0.0023, unpaired two-tailed Student's t-test). The lack of improvement of survival when magneto-EVs were injected at 3 and 24 hours is likely due to the rapid progression of kidney injury in the LPS-AKI model. Indeed, without treatment, the animal loss at 24 and 48 hours was 50 and 100%, respectively.

To assess whether the accumulation of magneto-EVs in the injured kidney and their subsequent protective effect is attributed to their stem cell origin, we also isolated EVs from fetal bovine serum (FBS) and injected them into LPS-AKI mice using the same protocol as for iPSC-EVs. The ex vivo MRI results (FIG. 4J) showed that a high quantity of FBS-EVs accumulated in the cortex similarly to iPSC-EVs, suggesting that the accumulation of magneto-EVs in the injured sites may not be specific to their cell origin. However, despite their similar uptake pattern FBS-EVs did not generate protection against the LPS-induced kidney injury (FIG. 4K, P=0.2055), suggesting that the therapeutic effects are specific to the cell origin of the EVs.

Biodistribution of iPSC-EVs in Other Experimental Injury Models

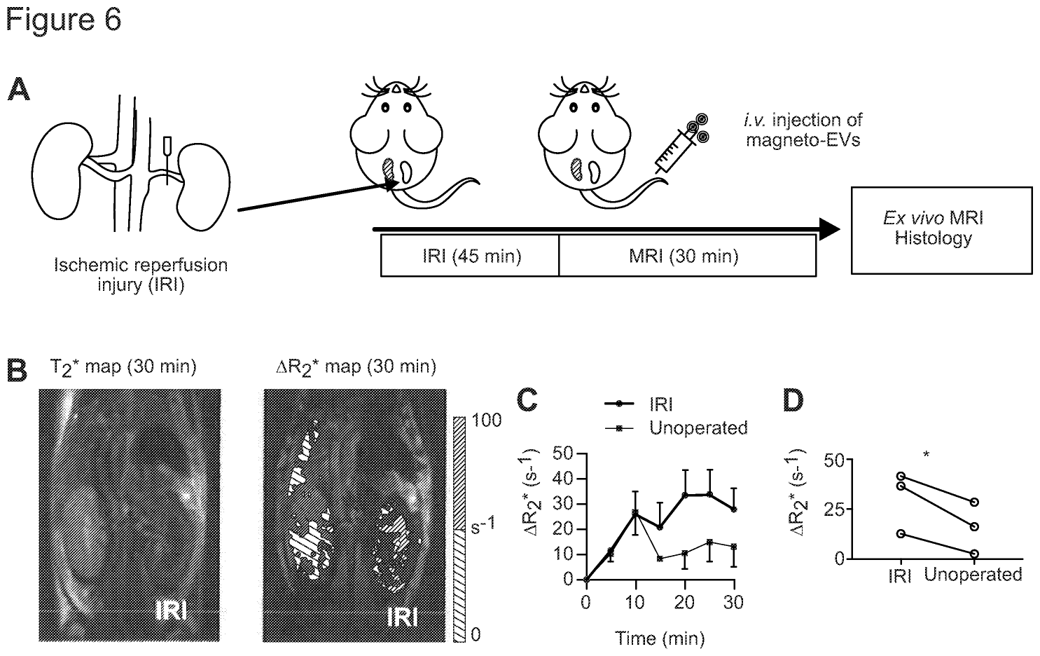

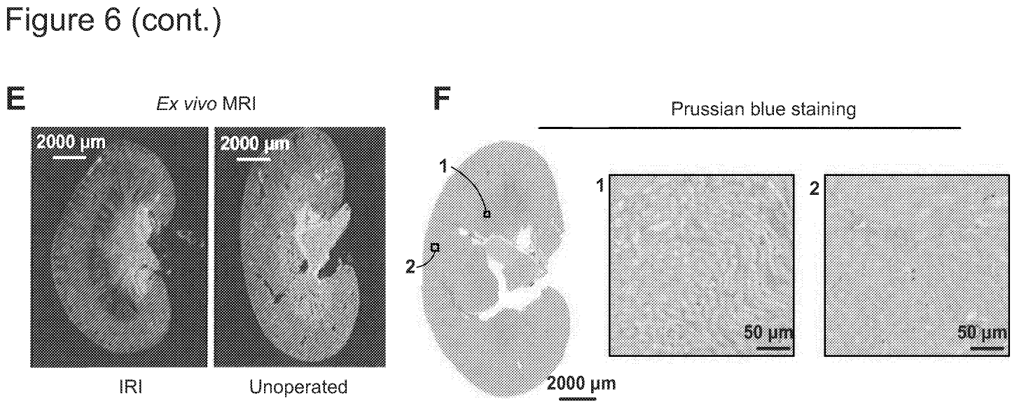

We further assessed the ability to track magneto-EVs in two other animal injury models. First, kidneys were subjected to a unilateral ischemia-reperfusion injury (IRI) (FIG. 6A). An acute injury in the right kidney was obtained by occluding the blood supply for 45 min, with the untreated left kidney as control. iPSC-derived magneto-EVs were administered i.v. at the same time when reperfusion started. As shown in FIGS. 6B,C, a higher EV uptake was observed in the injured kidneys but not the contralateral ones (ΔR₂*=30.1 s⁻¹ and 15.7 s⁻¹, P=0.04, two-tailed paired Student's t-test, n=4). The biodistribution pattern of magneto-EVs homing to the injury site was distinct from the AKI mice induced by LPS injection. Both the ex vivo MRI (FIG. 6E) and histology (FIG. 6F) showed more magneto-EVs accumulating in the medulla than the cortex, representing the difference in the primary site of injury between these two experimental models.

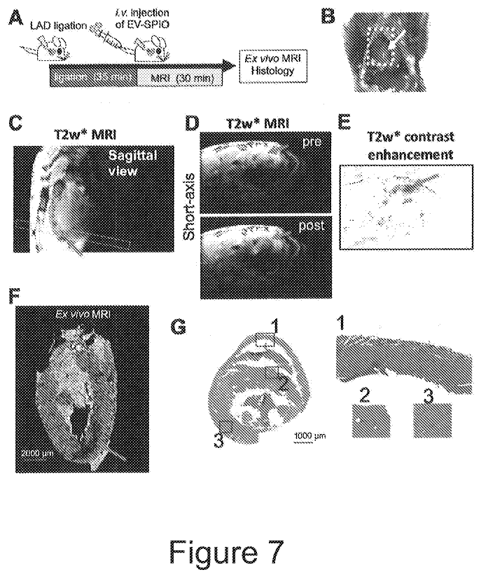

We then applied our technology to study the distribution of i.v.-injected magneto-EVs in an IR-injured mouse heart, which was induced through the ligation of the left anterior descending (LAD) coronary artery for 35 min, followed by reperfusion(30) (FIGS. 7A,B). The MRI results revealed that magneto-EVs accumulated selectively at the injury sites (FIGS. 7C,D). The distribution of EVs followed an alignment with the injured myocardium (FIG. 7E), which was confirmed by high resolution ex vivo MRI (FIG. 7F) and Prussian blue staining (FIG. 7G).

### Materials and Methods for Example

### Materials

Carboxyl SPIOs (SPIO—COOH, core diameter=5 nm) was purchased from Ocean Nanotech (Springdale, Ark.). Hexa-histidine peptide was purchased from GeneScript (Springdale, Ark., USA). 1-Ethyl-3-(3-dimethylaminopropyl)-carbodiimide (EDC), sulfo-N-hydroxysuccinimide (sulfo-NHS) and Ni-NTA were purchased from Sigma-Aldrich (St. Louis, Mo., USA).

### Cell Culture

Human iPSCs were programmed from blood cells of a healthy male donor(38), cultured in 6-well plates coated with vitronectin (Gibco, Calsbad, Calif.), and maintained in Essential 8 (E8) medium (Gibco) supplemented with 10 μM Y-27632 dihydrochloride (Stem Cell Technologies, Vancouver, Canada at 37° C. in a humidified atmosphere of 5% CO₂ and 95% air. The culture medium was changed daily after gently washing the cells with 10 mM PBS, pH=7.4. Cells were passaged at 80%-90% confluence using TrypLE™ Express Enzyme (Gibco) supplemented with 10 μM Y-27632 dihydrochloride. Cells were routinely checked for mycoplasma contamination.

### Synthesis of SPIO-His

Fifty microliters of SPIO-carboxyl (2 mg/mL) was mixed with 100 μg EDC (10 mg/mL) and 100 μg NHS (10 mg/mL), and 100 μL MES buffer (pH=6.0). The solution was shaken for 30 min. One mg of peptide (25 mg/mL in dd H₂O) was then added, followed by adding 200 μL 10 mM PBS, pH=7.4. The pH of the solution was then adjusted to 7.4, followed by shaking for 2 h. The product was dialyzed, lyophilized and reconstituted to 2 mg Fe/mL.

### Collection, Purification and Enrichment of EVs

To acquire concentrated EVs, medium was harvested from multiple passages of iPSC culture and concentrated. Culture medium in 50 mL tubes was centrifuged for 10 min at 300 g followed by another 10 min at 2,000 g at 4° C. to remove cells and debris. The medium was then concentrated to 200 μL using an Amicon ultra-15 filter column and an Ultracel-100 membrane by centrifugation at 4,000 g for 20 min (MilliporeSigma, Billerica, Mass., USA). Then, 0.5 mL of the concentrated medium was loaded to a qEV column (iZON, Cambridge, Mass., USA) and eluted with PBS in 500 μL fractions with collection of the 7-9 fractions. This procedure removes small medium molecules from the concentrate. Purified EVs (about 2.4 mL elute from a total of 300 mL medium) were further concentrated six times using a qEV column to a final volume of 0.4 mL. To further enrich magneto-EVs, the microcentrifuge tube containing purified magneto-EVs was fixed upright on a 1-inch cube Neodymium magnet (CMS magnetics, Garland, Tex., USA) overnight and the pelleted magneto-EVs were resuspended in the desirable volume of PBS. Isolation and purification of FBS (catalog #F2442, Sigma-Aldrich) derived EVs were performed in a similar manner.

### Electroporation of EVs

Fifty microliters of concentrated EVs (1.1×10¹¹/mL) were mixed with 25 μL of 2 mg/mL SPIO-His. Electroporation was performed with a Gene Pulser Xcell Electroporation Systems (Biorad) using two pulses of 240 V/mm and 100 F Capacitance with a 1 mm cuvette. EVs were then transferred to a clean microcentrifuge tube and placed on ice for 1 h before Ni-NTA purification.

### Purification of Magneto-EVs

Ni-NTA columns were prepared by packing 1 mL Ni-NTA His·Bind resins (Sigma) into a 6-ml ISOLUTE® Single Fritted Reservoir column with 10 μm polyethylene frit (Biotage, Charlotte, N.C., USA), followed by washing with 5 mL PBS. After electroporation, EVs (˜75 μL) were resuspended in 200 μL PBS and then loaded onto a Ni-NTA column and gently shaken for 30 min. After the first elute was collected, the column was rinsed with 200 μL PBS. All elutes were collected using 1 mL microcentrifuge tubes.

The size and iron content of EVs was measured by dynamic light scattering (DLS, Nanosizer ZS90, Malvern Instruments) and by inductively coupled plasma optical emission spectroscopy (ICP-OES, Thermo iCAP 7600, ThermoFisher Scientific, Waltham, Mass., USA), respectively. To estimate the loss of EVs during the purification procedure, the numbers of EVs before and after purification were measured using a nanoparticle tracking analysis (NTA) instrument (Zetaview, Particle Metrix, Germany) using a 488-nm laser and ZetaView 8.04.02 software.

### Transmission Electron Tomography

Carbon-coated 400 mesh copper grids (Electron Microscopy Services, Hatfield, Pa., USA) were placed on 30 μl drops of SPIO or EV samples for one minute. Grids were quickly washed with two drops of dH₂O and blotted dry on a filter paper. Grids were stained for one minute with 2% uranyl acetate (Electron Microscopy Services) in dH₂O and blotted dry. All imaging was performed with a Zeiss Libra 120 TEM operated at 120 KV and equipped with an Olympus Veleta camera (Olympus Soft Imaging Solutions GmbH, Münster, Germany).

### Mouse Models of Acute Kidney Injury

All animal experiments were approved by our Animal Care and Use Committee. Male C57BL/6J mice (6-8 weeks of age) were acquired from Jackson Laboratories (Bar Harbor, Me., USA). The LPS-AKI model was established as described previously(25) by intraperitoneal (i.p.) administration of lipopolysaccharide (LPS, Sigma Aldrich) at a dose of 10 mg/kg. The IRI-AKI model was established according to a previously published procedure(39). In brief, animals were anesthetized with 2% isoflurane and an incision was made in the back muscle and skin layer to expose the right kidney and the renal vascular pedicle was clamped using a microvessel clamp (#18052-03, Fine Science Tools, Foster City, Calif., USA) for 45 min, followed by suture-closing the incisions.

### Mouse Model of Heart Ischemic and Reperfusion Injury (IRI)

Male C57BL/6J mice under induction anesthesia with 3-4% isoflurane received 0.03-0.07 mg/kg buprenorphine subcutaneously (s.c.). Isoflurane was then adjusted to 1-2% and 2 mg/kg succinylcholine was administered i.p. After 5 min, a left thoracotomy was performed in the 5th to 6th intercostal space. The pericardium was torn, and the coronary artery was located. A piece of 7-0 prolene was tied around the coronary artery with a small piece of black polyethylene PE10 tubing (Braintree Scientific Inc., Braintree, Mass., USA) under the suture. Occlusion was performed for 35 min. During occlusion, ribs were closed with one single 5-0 silk suture and the skin was closed with a bulldog clamp (Fine Science Tools, Foster City, Calif., USA). At 5 min prior to ending the occlusion, the chest was reopened carefully and the sutures were removed. Ribs and skin were closed with 5-0 silk. Mice were allowed to regain consciousness and a second dose of buprenorphine at the dose of 0.06-0.075 mg/kg was administered s.c.

### In Vitro MRI

Samples of SPIO—COOH, SPIO-His, unlabeled and labeled EVs were prepared at different concentrations in 10 mM PBS, pH=7.4 and transferred to 5 mm glass NMR tubes, and then combined for MRI measurements on a Bruker 9.4 Tesla vertical bore scanner equipped with a 20 mm birdcage transmit/receive coil. T₂ relaxation times were measured using the Carr-Purcell-Meiboom-Gill (CPMG) method at room temperature as previously described(40). The acquisition parameters were: TR/TE=25 s/4.3 ms, RARE factor=16, matrix size=64×64, in-plane resolution=0.25×0.25 mm and slice thickness=2 mm. Each T₂w image took approximately 1′40″ to acquire. The T₂ relaxivities (r₂) were calculated based on mean R₂(=1/T₂) of each sample and their concentrations (c), using the following equation:

R₂=R₂⁰+r₂×c
Where R₂⁰ represents the inherent water proton transverse relaxation rate.

### In Vivo MRI

All animal studies were performed on a 11.7T Biospec (Bruker) horizontal bore scanner equipped with a mouse brain surface array RF coil (receiver) and a 72 mm volume coil (transmitter). A 30-min dynamic scan was acquired using a fast low angle shot (FLASH) gradient echo sequence immediately before i.v. injection of 1×10⁹ magneto-EVs or 1.5 μg SPIO-His (having the same iron amount as that in magneto-EVs) in 200 μL PBS. The acquisition parameters were: flip angle=25°, TR=800 ms, TE=5.8 ms, matrix size=256×128 and resolution=167×280 mm². Before and 30 min after injection T₂* maps were also acquired using a multiple gradient echo (MGE) pulse sequence with TR=800 ms and TE times of 2.6, 5.8, 9, 12.2, 15.4, 18.6, 21.8, and 25 ms.

After MRI, mice were euthanized by cervical dislocation under anesthesia and tissues of interest were collected and fixed in 4% paraformaldehyde solution for ex vivo MRI and histological analysis.

### Ex Vivo MRI

Excised organs (i.e., kidney, heart, and liver) were transferred to a 5 ml syringe filled with proton-free fluid Fomblin (Solvay Solexis, Inc., USA) and ex vivo high-resolution MRI was performed on a vertical bore 9.4T Bruker scanner equipped with a 15 mm birdcage transmit/receive volume coil. A three-dimensional FLASH sequence was used with TE=6 ms, TR=150 ms, matrix size=310×230×155, FOV=12×9×6 mm, resolution=0.039×0.039×0.039 mm, averages=5, and flip angle=15°. The total scan time was 6 h 43 m 12 s.Amira 3D Visualization Software 5.4.3 (Visage Imaging Inc., Carlsbad, Calif., USA) was used to quantify the areas of hypointense signal and to visualize the 3D distribution of magneto-EVs.

### Histological Analysis

Excised tissues were paraffin-embedded and sectioned at 5 μm thickness, and stained with Prussian blue and Periodic acid-Schiff (PAS). Sections were imaged using a Zeiss Axio Observer Z1 microscope (Zeiss, Oberkochen, Germany) and processed using Zen Pro software.

Effects of iPSC-EV Treatment on LPS-Induced AKI Mice.

Five treatment groups were tested: 1) iPSC-EV was administered at the same time as LPS injection; 2) iPSC-EV was administered at 3 hours after LPS injection; 3) iPSC-EV was administered at 24 hours after LPS injection; 4) FSB-EV was administered at 3 hours after LPS injection; and 5) vehicle control (200 μμL PBS) was administered at the same time as LPS injection. Five mice were randomly chosen for each group. For all EV treatment groups, 2×10⁹ EVs in 200 μμL PBS were administered i.v. through the tail vein. Animal survival was monitored for each group every day up to 6 days.

### Statistical Analysis

All data are presented as mean±s.e.m. GraphPad Prism version 8 (GraphPad Software Inc., San Diego, Calif., USA) was used to perform statistical analysis. A unpaired two-tailed Student's t-test was used to compare the difference between two groups. Differences with P<0.05 were considered statistically significant. The Kaplan-Meier method was used to analyze animal survival data.

### REFERENCES FOR EXAMPLE

1. D. Karpman, A. L. Stahl, I. Arvidsson, Extracellular vesicles in renal disease. Nature reviews. Nephrology 13, 545 (September, 2017).
2. G. van Niel, G. D'Angelo, G. Raposo, Shedding light on the cell biology of extracellular vesicles. Nat Rev Mol Cell Biol 19, 213 (April, 2018).
3. M. Colombo, G. Raposo, C. Thery, Biogenesis, secretion, and intercellular interactions of exosomes and other extracellular vesicles. Annu Rev Cell Dev Bi 30, 255 (2014).
4. M. Gnecchi, Z. Zhang, A. Ni, V. J. Dzau, Paracrine mechanisms in adult stem cell signaling and therapy. Circ Res 103, 1204 (Nov. 21, 2008).
5. L. Chen, E. E. Tredget, P. Y. Wu, Y. Wu, Paracrine factors of mesenchymal stem cells recruit macrophages and endothelial lineage cells and enhance wound healing. PLoS One 3, e1886 (Apr. 2, 2008).
6. S. Rani, A. E. Ryan, M. D. Griffin, T. Ritter, Mesenchymal Stem Cell-derived Extracellular Vesicles: Toward Cell-free Therapeutic Applications. Mol Ther 23, 812 (May, 2015).
7. I. Vishnubhatla, R. Corteling, L. Stevanato, C. Hicks, J. Sinden, The Development of Stem Cell-Derived Exosomes as a Cell-Free Regenerative Medicine. Journal of Circulating Biomarkers 3, (2014).
8. F. Collino et al., AKI Recovery Induced by Mesenchymal Stromal Cell-Derived Extracellular Vesicles Carrying MicroRNAs. Journal of the American Society of Nephrology: JASN 26, 2349 (October, 2015).
9. S. Bruno et al., Microvesicles derived from mesenchymal stem cells enhance survival in a lethal model of acute kidney injury. PloS one 7, e33115 (Mar. 14, 2012).
10. M. Adamiak et aL, Induced Pluripotent Stem Cell (iPSC)-Derived Extracellular Vesicles Are Safer and More Effective for Cardiac Repair Than iPSCs. Circ Res 122, 296 (Jan. 19, 2018).
11. B. Liu et aL, Cardiac recovery via extended cell-free delivery of extracellular vesicles secreted by cardiomyocytes derived from induced pluripotent stem cells. Nat Biomed Eng 2, 293 (May, 2018).
12. C. Grange et aL, Biodistribution of mesenchymal stem cell-derived extracellular vesicles in a model of acute kidney injury monitored by optical imaging. International journal of molecular medicine 33, 1055 (May, 2014).
13. O. P. Wiklander et aL, Extracellular vesicle in vivo biodistribution is determined by cell source, route of administration and targeting. J Extracell Vesicles 4, 26316 (2015).
14. T. Imai et aL, Macrophage-dependent clearance of systemically administered B16BL6-derived exosomes from the blood circulation in mice. J Extracell Vesicles 4, 26238 (2015).
15. D. W. Hwang et aL, Noninvasive imaging of radiolabeled exosome-mimetic nanovesicle using (99m)Tc-HMPAO. Sci Rep 5, 15636 (Oct. 26, 2015).
16. S. Shi et aL, Copper-64 Labeled PEGylated Exosomes for In Vivo Positron Emission Tomography and Enhanced Tumor Retention. Bioconjug Chem 30, 2675 (Oct. 16, 2019).
17. O. Betzer, N. Perets, E. Barnoy, D. Offen, R. Popovtzer, Labeling and tracking exosomes within the brain using gold nanoparticles. Proc Spie 10506, (2018).
18. A. Busato et al., Magnetic resonance imaging of ultrasmall superparamagnetic iron oxide-labeled exosomes from stem cells: a new method to obtain labeled exosomes. International journal of nanomedicine 11, 2481 (2016).
19. L. Hu, S. A. Wickline, J. L. Hood, Magnetic resonance imaging of melanoma exosomes in lymph nodes. Magnetic resonance in medicine 74, 266 (July, 2015).
20. K. O. Jung, H. Jo, J. H. Yu, S. S. Gambhir, G. Pratx, Development and MPI tracking of novel hypoxia-targeted theranostic exosomes. Biomaterials 177, 139 (September, 2018).
21. P. Y. Lee et aL, Induced pluripotent stem cells without c-Myc attenuate acute kidney injury via downregulating the signaling of oxidative stress and inflammation in ischemia-reperfusion rats. Cell Transplant 21, 2569 (2012).
22. S. Liu et al., Highly Purified Human Extracellular Vesicles Produced by Stem Cells Alleviate Aging Cellular Phenotypes of Senescent Human Cells. Stem Cells 37, 779 (June, 2019).
23. J. H. Jung, X. Fu, P. C. Yang, Exosomes Generated From iPSC-Derivatives: New Direction for Stem Cell Therapy in Human Heart Diseases. Circ Res 120, 407 (Jan. 20, 2017).
24. H. Ittrich et. al., In vivo magnetic resonance imaging of iron oxide-labeled, arterially-injected mesenchymal stem cells in kidneys of rats with acute ischemic kidney injury: detection and monitoring at 3T. J Magn Reson Imaging 25, 1179 (June, 2007).
25. J. Liu et al., CEST MRI of sepsis-induced acute kidney injury. NMR Biomed 31, e3942 (August, 2018).
26. K. Doi, A. Leelahavanichkul, P. S. Yuen, R. A. Star, Animal models of sepsis and sepsis-induced kidney injury. J Clin Invest 119, 2868 (October, 2009).
27. M. Morishita, Y. Takahashi, M. Nishikawa, Y. Takakura, Pharmacokinetics of Exosomes-An Important Factor for Elucidating the Biological Roles of Exosomes and for the Development of Exosome-Based Therapeutics. J Pharm Sci 106, 2265 (September, 2017).
28. Y. Feng et al., Improved MRI R2* relaxometry of iron-loaded liver with noise correction. Magnetic resonance in medicine 70, 1765 (December, 2013).
29. L. Zhang et al., Nerolidol Protects Against LPS-induced Acute Kidney Injury via Inhibiting TLR4/NF-kappaB Signaling. Phytother Res 31, 459 (March, 2017).
30. J. M. J. Pickard, N. Burke, S. M. Davidson, D. M. Yellon, Intrinsic cardiac ganglia and acetylcholine are important in the mechanism of ischaemic preconditioning. Basic Res Cardiol 112, 11 (March, 2017).
31. J. W. Bulte, In vivo MRI cell tracking: clinical studies. AJR Am J Roentgenol 193, 314 (August, 2009).
32. I. J. de Vries et al., Magnetic resonance tracking of dendritic cells in melanoma patients for monitoring of cellular therapy. Nat Biotechnol 23, 1407 (November, 2005).
33. J. W. Bulte, D. L. Kraitchman, Iron oxide MR contrast agents for molecular and cellular imaging. NMR Biomed 17, 484 (November, 2004).
34. J. P. Laissy et al., Reversibility of experimental acute renal failure in rats: assessment with USPIO-enhanced MR imaging. J Magn Reson Imaging 12, 278 (August, 2000).
35. F. Pi et aL, Nanoparticle orientation to control RNA loading and ligand display on extracellular vesicles for cancer regression. Nat Nanotechnol 13, 82 (January, 2018).
36. J. L. Hood, M. J. Scott, S. A. Wickline, Maximizing exosome colloidal stability following electroporation. Anal Biochem 448, 41 (Mar. 1, 2014).
37. Y. Zhou et al., Exosomes released by human umbilical cord mesenchymal stem cells protect against cisplatin-induced renal oxidative stress and apoptosis in vivo and in vitro. Stem cell research & therapy 4, 34 (Apr. 25, 2013).
38. B. K. Chou et al., Efficient human iPS cell derivation by a non-integrating plasmid from blood cells with unique epigenetic and gene expression signatures. Cell Res 21, 518 (March, 2011).
39. Q. Wei, Z. Dong, Mouse model of ischemic acute kidney injury: technical notes and tricks. Am J Physiol Renal Physiol 303, F1487 (Dec. 1, 2012).
40. J. Zhang et al., Triazoles as T2-Exchange Magnetic Resonance Imaging Contrast Agents for the Detection of Nitrilase Activity. Chemistry (Weinheim an der Bergstrasse, Germany) 24, 15013 (Oct. 9, 2018).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for manufacturing extracellular vesicles comprising sticky imaging particles comprising:
associating one or more sticky elements to one or more imaging particles to form one or more sticky imaging particles;
forming extracellular vesicles (EV) comprising one or more of the sticky imaging particles.

2. The method of claim 1 wherein one or more sticky imaging particles associated with one or more extracellular vesicles (EV) are formed.

3. The method of claim 1 wherein the sticky imaging particles are incubated with extracellular vesicles (EV) to form sticky imaging particles associated with extracellular vesicles (EV).

4. The method of claim 1 wherein the sticky imaging particles are incubated with extracellular vesicles (EV) to form sticky imaging particles encapsulated in extracellular vesicles (EV).

5. (canceled)

6. The method of claim 1 further comprising isolating sticky imaging particles encapsulated in EVs.

7. The method of claim 1 further comprising separating the sticky imaging particles encapsulated in EVs from sticky imaging particles that are encapsulated in EVs.

8-9. (canceled)

10. The method of claim 1 wherein sticky imaging particles not associated with extracellular vesicles (EV) adhere to a chromatography material and sticky imaging particles associated with extracellular vesicles (EV) adhere to the chromatography material comparatively less than sticky imaging particles not associated with extracellular vesicles (EV) to thereby enable separation of 1) the sticky imaging particles not associated with extracellular vesicles (EV) and 2) sticky imaging particles that are associated with extracellular vesicles (EV).

11. (canceled)

12. The method of claim 1 wherein the imaging particle is a supermagnetic iron oxide (SPIO) particle.

13. The method of claim 1 wherein the imaging particle is a nanoparticle.

14. The method of claim 1 wherein the sticky element is a peptide having two or more amino acid residues.

15. The method of claim 14 wherein the peptide comprises, consists essentially of or consists of histidine (HIS) amino acid residues.

16-19. (canceled)

20. A sticky imaging particle comprising:
an imaging particle; and
one or more sticky elements.

21. The stick imaging particle of claim 20 wherein the imaging particle is associated with a sticky element.

22. The sticky imaging particle of claim 21 wherein the imaging particle is covalently and/or non-covalently bonded to one or more sticky elements.

23. The sticky imaging particle of claim 20 wherein the imaging particle is magnetic.

24. The sticky imaging particle of claim 20 wherein the sticky element is a peptide comprising two or more amino acid residues.

25. (canceled)

26. The sticky imaging particle of claim 24 wherein the peptide consists of, consists essentially of or comprises 2 to 20 HIS amino acid residues.

27-28. (canceled)

29. An imaging composition comprising extracellular vesicles and one or more sticky imaging particles,
wherein the sticky imaging particles comprise an imaging particle and one or more sticky elements, and the composition has less than 40 weight % of sticky imaging particles that are not associated with extracellular vesicles, where weight % is based on total composition weight.

30. (canceled)

31. The imaging composition of claim 29 wherein the imaging composition further comprises one or more therapeutic agents.

32. A method of imaging comprising:
administering an imaging composition of claim 29 to a subject.

33-38. (canceled)
